(19) United States
(12) Patent Application Publication
(10) Pub. No.: US 2022/0033776 A1
(43) Pub. Date: Feb. 3, 2022

(54) METHODS OF MAKING PLATELETS COMPRISING MODIFIED RECEPTORS AND USES THEREOF

(71) Applicant: University of Utah Research Foundation, Salt Lake Ciry, UT (US)

(72) Inventor: Tara Deans, Salt Lake City, UT (US)

(21) Appl. No.: 17/280,415

(22) PCT Filed: Oct. 1, 2019
(86) PCT No.: PCT/US19/54032

§ 371 Date: Mar. 26, 2021

Related U.S. Application Data
(60) Provisional application No. 62/741,971, filed on Oct. 5, 2018.

(51) Int. Cl. C12N 5/078; C07K 14/705; C12N 15/90; A61K 35/19

(52) U.S. Cl. CPC C12N 5/0644; C07K 14/705; C12N 15/907; C12N 2506/11; C12N 2510/00; C12N 2830/008; A61K 35/19

(57) ABSTRACT

Disclosed herein are methods of producing platelets comprising a modified receptor, therapeutic agents, peptides, and/or bioactive molecules. The cells produced by the methods disclosed herein can be used to treat, manage, prevent and diagnosis, for example, lysosomal storage diseases, diabetes and cancer. The cells produced by the methods disclosed herein can be engineered to comprise receptors capable of activating platelets to trigger the release of enzymes, biomolecules or therapeutic agents upon binding to specific drugs and/or binding to tissue specific peptides.

### CROSS REFERENCE TO RELATED APPLICATIONS

This application claims the benefit of the filing date of U.S. Provisional Application 62/741,971, which was filed on Oct. 5, 2018. The content of this earlier filed application is hereby incorporated by reference herein in its entirety.

### INCORPORATION OF THE SEQUENCE LISTING

The present application contains a sequence listing that was submitted in ASCII format via EFS-Web concurrent with the filing of the application, containing the file name 21101_0377P1_Sequence_Listing which is 427 bytes in size, created on Sep. 26, 2019, and is herein incorporated by reference in its entirety.

### BACKGROUND

Lysosomal Storage Diseases (LSDs) are caused by defects in multiple aspects of lysosomal function, most commonly mutations in lysosomal hydrolases that are enzymes involved in the degradation of macromolecules¹. These mutations lead to defective enzymes that are involved in the degradation of cellular macromolecules. Macromolecules created during cellular metabolism must be broken down for either excretion or reuse; otherwise this metabolic waste overwhelms the cell's storage capacity, leading to cellular distortion, inactivation, and destruction. As cellular destruction becomes more widespread, tissue and eventual organ failure occur. Studies have shown that diseased cells are capable of taking up exogenous enzymes secreted by neighboring cells, and that even a small increase in residual enzyme activity can have a profound impact on restoring lysosomal function. This realization has led to the current treatments for LSDs that include the recombinant production of hydrolases and their infusion into the blood, in addition to bone marrow transplants that contain a population of cells with the capacity to produce the missing enzyme. However, protein instability prevents them from reaching major target organs at therapeutic doses, and risks associated with bone marrow transplantation (graft vs. host) prevent clinical efficacy⁶⁻¹⁰.

### SUMMARY

Disclosed herein are nucleic acid constructs comprising: a first genetic circuit comprising a tissue-specific promoter operatively linked to a sequence capable of encoding a modified receptor.

Disclosed herein are megakaryocytes comprising nucleic acid constructs, wherein the nucleic acid constructs comprise a first genetic circuit comprising a tissue-specific promoter operatively linked to a sequence capable of encoding a modified receptor.

Disclosed herein are engineered megakaryocytes comprising: a modified receptor.

Described herein are compositions and methods for engineering platelets with the ability to release bioactive biomolecules into tissues using controlled release to enhance the health and function of these tissues that may lead to improved life expectancy of these patients (e.g., patients with LSD). Also disclosed are engineering platelets with the ability to release bioactive molecules into relevant tissues using controlled release. Also disclosed are methods of making engineered platelets with the ability to release bioactive molecules into relevant tissues using controlled release mechanisms. Also, disclosed are engineering platelets with the ability to release bioactive biomolecules into relevant tissues using controlled release that comprise receptors capable of activating platelets to trigger the release of biomolecules upon binding to specific drugs and/or binding to tissue specific peptides.

Disclosed herein are methods of producing red blood cells or platelets, the method comprising: a) providing a genetically engineered feeder cell, wherein the feeder cell comprises one or more genetic circuits, wherein the one or more genetic circuits comprise one or more genes of interest; and one or more promoters; b) providing a genetically engineered fed cell, wherein the fed cell comprises one or more genetic circuits, wherein the one or more genetic circuits comprise one or more genes of interest, wherein the one or more genes of interest are different than the one or more genes of interest in a); and one or more promoters; and c) culturing the genetically engineered feeder cell in a) with the genetically engineered fed cell in b) in a media under conditions that permit the genetically engineered fed cells to differentiate into red blood cells or platelets; wherein one or more of the genetically engineered fed cells differentiate into red blood cells or platelets. Such methods can also include engineering the fed cells so that the red blood cells and platelets that differentiate from the fed cells comprise receptors capable of activating platelets to trigger the release of enzymes upon binding to specific drugs and/or binding to tissue specific peptides.

Disclosed herein are methods of producing platelets or red blood cells comprising a therapeutic agent, the method comprising a) providing a genetically engineered feeder cell, wherein the feeder cell comprises one or more genetic circuits; wherein the one or more genetic circuits comprise one or more genes of interest; and one or more promoters; b) providing a genetically engineered fed cell, wherein the fed cell comprises one or more genetic circuits; wherein the one or more genetic circuits comprise one or more genes of interest, wherein the one or more genes of interest are different than the one or more genes of interest in a); and one or more promoters; c) culturing the genetically engineered feeder cell in a) with the genetically engineered fed cell in b) in a media under conditions that permit the genetically engineered fed cells to differentiate into platelet and/or red blood cell progenitor stem cells; and d) producing the platelet or red blood cells comprising engineered receptors and/or a therapeutic agent. Such methods can also include engineering the platelets to comprise receptors capable of activating platelets to trigger the release of biomolecules upon binding to specific drugs and/or binding to tissue specific peptides.

### BRIEF DESCRIPTION OF THE DRAWINGS

FIGS. 1A-E show engineered platelets as delivery systems for disease treatments. FIG. 1A shows cellular distortion, inactivation and destruction in lysosomal storage diseases (LSD). FIG. 1B shows that megakaryocytes form platelets from their cytoplasmic extensions and that these formed platelets are filled with bioactive proteins. FIG. 1C shows engineered platelets filled with lysosomal enzymes. FIG. 1D shows non-engineered platelets are activated by thrombin and other small molecules. FIG. 1E shows that engineering designer receptors exclusively activated by designer drugs (DREADDs) on platelets so the receptor binds to pharmaceutically inert small molecules and no longer to the endogenous molecules.

FIG. 2 shows an overview of HSC differentiation. HSCs are multipotent stem cells that have the potential to differentiate into various precursor cells that become more specialized blood cells.

FIGS. 3A-D shows a method of assessing HSC growth and potential. FIG. 3A shows (i) cells isolated from mouse bone marrow, (ii) grown in Dexter culture for the specified days, (iii) cells were transferred to MethoCult, and (iv) the number of lineage-committed colonies were counted over time. FIG. 3B shows cells isolated from mouse bone marrow were (i) grown in MethoCult for 7 days and (ii) labeled with CD41 and CD45 to assess their differentiation. CD41 labels platelets (cells in P4 gate, pink), CD45 labels all nucleated cells of blood lineage (cells in the P3 gate, blue). Those cells labeled with both (cells in P2 gate, green) are MKs and progenitor cells. FIG. 3C shows LSK+ cells (HSCs) from the bone marrow were sorted and grown on OP9 stromal cells for 8 days. FIG. 3D shows ES cells were grown on OP9 stromal cells for 9 days, and LSK+ cells were detected by flow cytometry (cells in P3 gate, blue).

FIGS. 4A-D shows Landing pad in Rosa26 locus. FIG. 4A shows that 3× attP sites that were inserted into the Rosa26 allele in mouse ES cells using CRISPR technology. FIG. 4B shows that using PhiC31 integrase, the genetic circuits can target the Rosa26 allele for stable integration. FIG. 4C shows the results of PCR screen to confirm integration. FIG. 4D shows the PCR results of cDNA from mouse using screening primers. Lane 1: 2-log ladder, lane2: wild type with no integration, land 3: insertion of landing pad.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for cancer, such as, for example, prior to the administering step.

As used herein, the term “comprising” can include the aspects “consisting of” and “consisting essentially of.”

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “expression vector” is herein to refer to vectors that are capable of directing the expression of genes to which they are operatively-linked. Common expression vectors of utility in recombinant DNA techniques are often in the form of plasmids. Recombinant expression vectors can comprise a nucleic acid as disclosed herein in a form suitable for expression of the acid in a host cell. In other words, the recombinant expression vectors can include one or more regulatory elements or promoters, which can be selected based on the host cells used for expression that is operatively linked to the nucleic acid sequence to be expressed.

The term “sequence of interest” or “gene of interest” can mean a nucleic acid sequence (e.g., a therapeutic gene), that is partly or entirely heterologous, i.e., foreign, to a cell into which it is introduced.

The term “sequence of interest” or “gene of interest” can also mean a nucleic acid sequence, that is partly or entirely homologous to an endogenous gene of the cell into which it is introduced, but which is designed to be inserted into the genome of the cell in such a way as to alter the genome (e.g., it is inserted at a location which differs from that of the natural gene or its insertion results in “a knockout”). For example, a sequence of interest can be cDNA, DNA, or mRNA.

The term “sequence of interest” or “gene of interest” can also mean a nucleic acid sequence that is partly or entirely complementary to an endogenous gene of the cell into which it is introduced.

A “sequence of interest” or “gene of interest” can also include one or more transcriptional regulatory sequences and any other nucleic acid, such as introns, that may be necessary for optimal expression of a selected nucleic acid. A “protein of interest” means a peptide or polypeptide sequence (e.g., a therapeutic protein), that is expressed from a sequence of interest or gene of interest.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

The terms “alter” or “modulate” can be used interchangeable herein referring, for example, to the expression of a nucleotide sequence in a cell means that the level of expression of the nucleotide sequence in a cell after applying a method as described herein is different from its expression in the cell before applying the method.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

As used herein, “CRISPR system” and “CRISPR-Cas system” refers to transcripts and other elements involved in the expression of or directing the activity of CRISPR-associated (“Cas”) genes, including sequences encoding a Cas gene, a guide sequence (also referred to as a “spacer” in the context of an endogenous CRISPR system; e.g. guide RNA or gRNA), or other sequences and transcripts from a CRISPR locus. In some aspects, one or more elements of a CRISPR system is derived from a type I, type II, or type III CRISPR system. In some aspects, one or more elements of a CRISPR system are derived from a particular organism comprising an endogenous CRISPR system, such as Streptococcus pyogenes. Generally, a CRISPR system is characterized by elements that promote the formation of a CRISPR complex at the site of a target sequence (also referred to as a proto spacer in the context of an endogenous CRISPR system).

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, malady, disorder, sickness, illness, complaint, or affection.

As used herein, the terms “promoter,” “promoter element,” or “promoter sequence” are equivalents and as used herein, refers to a DNA sequence which when operatively linked to a nucleotide sequence of interest is capable of controlling the transcription of the nucleotide sequence of interest into mRNA. A promoter is typically, though not necessarily, located 5′ (i.e., upstream) of a nucleotide sequence of interest (e.g., proximal to the transcriptional start site of a structural gene) whose transcription into mRNA it controls, and provides a site for specific binding by RNA polymerase and other transcription factors for initiation of transcription.

Suitable promoters can be derived from genes of the host cells where expression should occur or from pathogens for this host cells (e.g., tissue promoters or pathogens like viruses). If a promoter is an inducible promoter, then the rate of transcription increases in response to an inducing agent. In contrast, the rate of transcription is not regulated by an inducing agent if the promoter is a constitutive promoter. Also, the promoter may be regulated in a tissue-specific or tissue preferred manner such that it is only active in transcribing the associated coding region in a specific tissue type(s) such as leaves, roots or meristem. The term “tissue specific” as it applies to a promoter refers to a promoter that is capable of directing selective expression of a nucleotide sequence or gene of interest to a specific type of tissue in the relative absence of expression of the same nucleotide sequence or gene of interest in a different type of tissue.

Although the foregoing invention has been described in some detail by way of illustration and example for purposes of clarity of understanding, certain changes and modifications may be practiced within the scope of the appended claims.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Platelets are anucleate blood cells that circulate throughout the body and play an important role in homeostasis, wound healing, angiogenesis, inflammation, and clot formation. Platelets are filled with secretory granules that store large amounts of proteins, which are formed from the cytoplasm of megakaryocytes (MKs), their precursor cells. When platelets are activated, bioactive proteins (e.g., biomolecules) are released from their granules to participate in a myriad of physiological processes. By taking advantage of platelets' innate storage, trafficking, and release capacities, they can be engineered to be delivery vehicles for the development of biomolecules for diseases or disorders, for example, for metabolic disorders. As disclosed herein, in some aspects blood platelets can be engineered to control the secretion of, for example, enzymes required for proper metabolic function serving as a therapeutic treatment for patients with various disorders including but not limited to Lysosomal Storage Diseases (LSDs).

Lysosomal Storage Diseases are caused by defects in multiple aspects of lysosomal function, most commonly mutations in lysosomal hydrolases that are enzymes involved in the degradation of macromolecules. These mutations lead to defective enzymes that are involved in the degradation of cellular macromolecules. Macromolecules created during cellular metabolism must be broken down for either excretion or reuse; otherwise this metabolic waste overwhelms the cell's storage capacity, leading to cellular distortion, inactivation, and destruction. As cellular destruction becomes more widespread, tissue and eventual organ failure occur. Studies have shown that diseased cells are capable of taking up exogenous enzymes secreted by neighboring cells, and that even a small increase in residual enzyme activity can have a profound impact on restoring lysosomal function. This realization has led to the current treatments for LSDs that include the recombinant production of hydrolases and their infusion into the blood, in addition to bone marrow transplants that contain a population of cells with the capacity to produce the missing enzyme. However, protein instability prevents them from reaching major target organs at therapeutic doses, and risks associated with bone marrow transplantation (graft vs. host) prevent clinical efficacy. As disclosed herein, genetic tools and circuits can be used to build and re-engineer cells to perform specific tasks that can be used, for example, for packaging lysosomal enzymes into platelets.

Also disclosed herein methods that include designing receptors that are capable of activating platelets to trigger the release of enzymes (or biomolecules or therapeutic agents) upon binding to specific drugs and/or binding to tissue specific peptides. As such, the platelets can be engineered as either systemic delivery devices or targeted tissue/organ specific delivery devices. The methods and the therapies disclosed herein can provide flexibility, precision, and personalization to patient treatment. The methods disclosed herein included the genetic tools and design principles described herein can serve as a general platform that can be combined with any other diseases/disorders for efficient and effective treatments alone as well as to complement existing therapies.

Also disclosed herein are methods of using platelets as delivery systems for treating a disease, disorder or condition. Also, disclosed herein are methods of using platelet-based therapeutic cell treatments for metabolic diseases, for example, lysosomal storage diseases (LSDs). As mentioned herein, LSDs are inherited metabolic diseases that are characterized by an abnormal buildup of various toxic materials in the body's cells as a result of lysosomal enzyme deficiencies¹⁻³. These malfunctioning enzymes represent a group of about 50 different genetic diseases and, though individually rare, their combined prevalence is estimated to be 1 in every 8,000 births. LSDs affect different parts of the body including the skeleton, brain, skin, heart, and central nervous system. Patients with LSD have a limited life expectancy. An important unmet clinical need for these metabolic diseases is an effective method for sustained delivery of lysosomal enzymes and for therapeutic levels of these enzymes to be delivered to the needed organ.

Further disclosed herein are methods of using engineered stem cells to create platelets that function as a delivery system for treating diseases or disorder (e.g. LSDs). Platelets are filled with secretory granules that store large amounts of proteins, which are formed from the cytoplasm of megakaryocytes (MKs), their precursor cells^{4,5}. Because platelets are cytoplasmic blebs made from extensions of MKs, they are filled with proteins present in the MK cytoplasm and do not contain a nucleus. Therefore, the genetic engineering that is done to the precursor stem cells will no longer exist in the platelets, making the disclosed method a practical therapeutic option for treating metabolic disorders.

Disclosed herein are gene circuits that have been created to probe stem cell fate decisions that can be used in genetically interactive cell culturing systems for improved platelet differentiation and isolation in vitro. Disclosed herein are methods to develop, refine, and integrate these technologies to reprogram platelets to function as a delivery system that can maintain control of when and where they release their therapeutic payload. In some aspects, the payload can be a therapeutic agent. In some aspects, the payload can be one or more endogenous biomolecules.

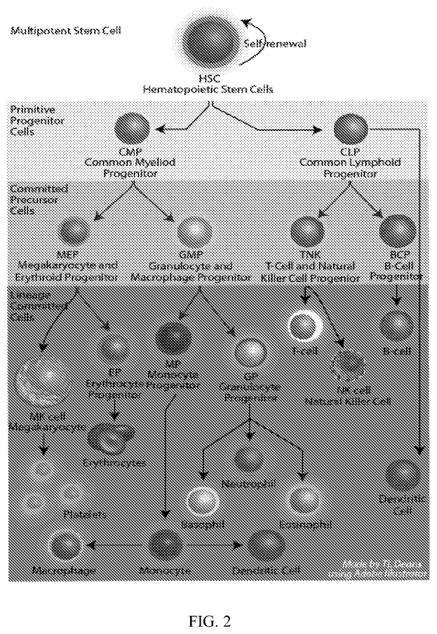

In the bone marrow, platelets are derived from the process of hematopoiesis, the differentiation of hematopoietic stem cells (HSCs) into specialized blood and immune cells (FIG. 2)¹¹. Platelets circulate in large numbers throughout the body with an average lifespan of 9-10 days, and the source of this large cell population is from MKs. In general, platelets are in a resting, inactive, state and require a trigger before becoming activated. Upon activation, platelets secrete more than 300 active biomolecules from their intracellular granules. Therefore, platelets possess many characteristics that make them attractive candidates for in vivo delivery of a variety of payloads: 1) they have extensive circulation range in the body, 2) they are anucleate cells, 3) they are biocompatible, 4) their average lifespan in humans is about 10 days, and 5) following activation, their protein granules serve as secretory vesicles, releasing components into the extracellular fluid.

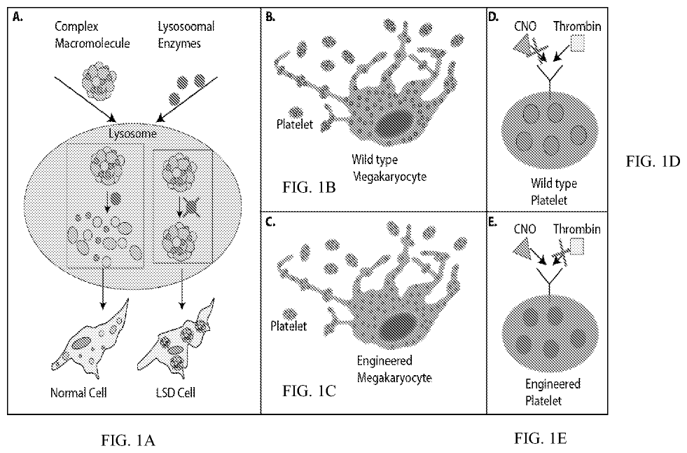

Disclosed herein are methods of using platelets as delivery systems for disease treatments. Also, disclosed herein, are platelet-based therapeutic cell treatments for metabolic diseases, including, for example, lysosomal storage diseases. LSDs are inherited metabolic diseases that are characterized by an abnormal buildup of various toxic materials in the body's cells as a result of lysosomal enzyme deficiencies¹⁻³ (FIG. 1A). These malfunctioning enzymes represent a group of about 50 different genetic diseases and, though individually rare, their combined prevalence is estimated to be 1 in every 8,000 births. LSDs affect different parts of the body including the skeleton, brain, skin, heart, and central nervous system. Patients with LSD have a limited life expectancy.

The current standard of care for treating LSDs is enzyme replacement therapy (ERT)⁷. For this treatment, enzymes are made recombinantly and their administration usually takes place through weekly infusions that can take up to three hours, although more frequent administrations have been seen for some ERTs⁷. The efficacy of many of these therapies is limited, however, due to the rapid degradation of exogenously injected enzymes, and their inability to reach major target organs at therapeutic doses to rectify disease. Therefore, there exists a substantial unmet clinical need for the development of therapies that address the limitations of injecting active enzymes directly into the bloodstream.

Described herein is the development of gene networks that can be used to direct stem cell differentiation to produce platelets as well as computational modeling and computer simulations used to develop genetic tools for platelets to control when and where they release their therapeutic payload, thus reprogramming the spatial and temporal activity of platelets. In some aspects, the platelets can be loaded with bioactive proteins. In some aspects, the platelets comprise endogenous bioactive proteins. While the initial experiments used mouse models, the methods described herein can be used to reprogram platelets as delivery systems that can be used in any mammalian system, including humans. While some of the mechanisms of mouse and human platelet biology differ, establishing this technology in mouse cells, will allow the application of these technologies to human cells (e.g., iPS cells), along with some adjustments to account for these differences.

Synthetic biology can be used as a research tool. The emerging field of synthetic biology has produced an exciting toolbox of genetic regulatory systems, and can be used to build new genetic circuits to implement control and specific functions in mammalian cells for the purpose of applying these tools in basic research and for therapeutic applications. The complexity of cell signaling networks can be simplified by considering genetic networks composed of subsets of simpler parts, or modules. This simplification is the foundation of synthetic biology, where engineering paradigms are applied in rational and systematic ways to produce predictable and robust systems for understanding or controlling cellular function^{15,16}. Ultimately this approach entails reprogramming cells to perform in predictable ways^{17,18}. Towards this end, genetic circuits have been built out of DNA and RNA that enable cells to perform Boolean logic functions ranging from memory¹⁹, and mathematical computations²⁰ to higher-order cellular functions like cancer cell identification²¹, controlling T cell populations²², and reporting on the microenvironment²³. The engineered gene circuits underlying these functions include genetic switches, oscillators, digital logic gates, and cell counters and have been designed to regulate gene expression in dynamic and predictable ways²⁴⁻²⁶. As described herein, synthetic biology tools can be used to mimic and regulate the intrinsic and extrinsic mechanisms that regulate HSC proliferation and differentiation into MKs for enhanced platelet production in an in vitro setting.

Compositions

Genetic circuits. Disclosed herein are genetic circuits. Disclosed herein are nucleic acid constructs comprising one or more genetic circuits. Any combination of the genetic circuits disclosed herein can be present in a single nucleic acid construct. Any of the genetic circuits disclosed herein can be described as a “first genetic circuit”, “second genetic circuit”, or a “third genetic circuit”.

In some aspects, a genetic circuit can comprise a tissue-specific promoter operatively linked to a sequence capable of encoding a modified receptor. In some aspects, a genetic circuit can comprise a tissue-specific promoter operatively linked to a sequence capable of encoding a modified receptor; and a gene of interest.

In some aspects, a genetic circuit can comprise one or more megakaryocyte differentiation genes. In some aspects, a genetic circuit can comprise one or more megakaryocyte differentiation genes; and a gene of interest. In some aspects, a genetic circuit can comprise a promoter operatively linked to the one more megakaryocyte differentiation genes. In some aspects, a genetic circuit can comprise a promoter operatively linked to the one more megakaryocyte differentiation genes and a gene of interest.

In some aspects, a genetic circuit can comprise a gene of interest.

Disclosed herein are nucleic acid constructs comprising: a first genetic circuit comprising a tissue-specific promoter operatively linked to a sequence capable of encoding a modified receptor. In some aspects, the tissue-specific promoter can be a megakaryocyte-specific promoter. In some aspects, the megakaryocyte-specific promoter can be a promoter that is specific for a particular development stage of the megakaryocyte as the megakaryocyte matures (i.e., becomes active). In some aspects, the megakaryocyte-specific promoter can be a human megakaryocyte promoter. In some aspects, the megakaryocyte-specific promoter can be CXCL4, GPIIb, or PTPRC. CXCL4 gene (chemokine (C-X-C motif) ligand 4, also known as platelet factor 4 (PF4)) is a small cytokine released from the alpha granules of activated platelets during platelet aggregation. CXCL4 promotes blood coagulation by moderating the effects of heparin-like molecules. GPIIb (glycoprotein IIb of the GPIIb/IIa complex, also known as CD41) is a part of an integrin complex found on the surface of platelets that act as a receptor for fibrinogen and von Willebrand factor. GPIIb aids in platelet activation and is important for normal platelet aggregation and endothelial adherence. PTPRC (protein tyrosine phosphatase, receptor type C, also known as CD45 or leukocyte common antigen) is a type I transmembrane protein that participates in a variety of cellular functions. In some aspects, the promoter can be regulatable. In some aspects, the promoter can be constitutively active.

As used herein, the term “promoter” refers to regulatory elements, promoters, promoter enhancers, internal ribosomal entry sites (IRES) and other elements that are capable of controlling expression (e.g., transcription termination signals, including but not limited to polyadenylation signals and poly-U sequences). Promoters can direct constitutive expression. Promoters can also direct expression in a temporal-dependent manner including but not limited to cell-cycle dependent or developmental stage-dependent. Examples of promoters include but are not limited to WPRE, CMV enhancers, and SV40 enhancers. Specific gene specific promoters can be used. Such promoters allow cell specific expression or expression tied to specific pathways. Any promoter that is active in mammalian cells can be used. In some aspects, the promoter is an inducible promoter including, but not limited to, Tet-on and Tet-off systems. Such inducible promoters can be used to control the timing of the desired expression. In some aspects, the promoter can be an inducible promoter. Examples of inducible promoters include but are not limited to tetracycline inducible system (tet); heat shock promoters and IPTG activated promoters. In some aspects, promoters are bidirectional.

The promoter and/or enhancer can be specifically activated either by light or specific chemical events which trigger their function. Systems can be regulated by reagents such as tetracycline and dexamethasone.

In some aspects, the genetic circuits as disclosed herein can comprise a promoter, for example but not limited to, enhancers, 5′ untranslated regions (5′UTR), 3′ untranslated regions (3′UTR), and repressor sequences; constitutive promoters, inducible promoter; tissue specific promoter, cell-specific promoter or variants thereof. Examples of tissue-specific promoters include, but are not limited to, albumin, lymphoid specific promoters, T-cell promoters, neurofilament promoter, pancreas specific promoters, milk whey promoter; hox promoters, a-fetoprotein promoter, human LIMK2 gene promoters, FAB promoter, insulin gene promoter, transphyretin, alpha.1-antitrypsin, plasminogen activator inhibitor type 1 (PAI-1), apolipoprotein myelin basic protein (MBP) gene, GFAP promoter, OPSIN promoter, NSE, Her2, erb2, and fragments and derivatives thereof. Examples of other promoters include, but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and variants thereof.

Disclosed herein are receptors that can be modified to be solely activated by artificial or exogenous agonists, referred to herein as a “modified receptor” or a “DREADD”. Receptors modified in this way are known to one of ordinary skill in the art using a technology called Designer Receptors Exclusively Activated by Designer Drugs (DREADD). Combining DREADD technology to engineer a megakaryocyte and/or a platelet to prepare the engineered megakaryocytes and/or engineered platelets is described herein. A receptor can be modified such that it is mutated to render it insensitive to endogenous ligands but sensitive to a substance that normally has no effect. One of ordinary skill in the art can provide or design such a modified receptor using known methods, and in view of the instant disclosure, apply them to the compositions and methods disclosed herein. The terms “modified receptor” and “DREADD” can be used interchangeably. A modified receptor (e.g., GPCR, PAR) can have a decreased binding affinity for a selected natural (e.g., endogenous) ligand of the modified receptor (relative to binding of the selected ligand by a wild-type receptor (e.g., GPCR, PAR)), but having normal, near normal, or preferably enhanced binding affinity for an exogenous, typically synthetic, ligand (e.g., a peptide or small molecule). Thus, modified receptor-mediated activation of modified receptor-expressing cells does not occur to a significant extent in vivo in the presence of the natural ligand, but responds significantly upon exposure to an exogenously introduced ligand (e.g., agonist). For example, the modified receptor can be superiorly activated by an exogenous ligand as compared to the natural ligand (e.g., activated to a greater or more significant extent by binding of the ligand than by binding to a selected natural or endogenous ligand at a similar concentration).

“Natural ligand” and “naturally occurring ligand” and “endogenous ligand” of a native GPCR can be used interchangeably herein to mean a biomolecule endogenous to a mammalian host, wherein the biomolecule binds to a native GPCR to elicit a G protein-coupled cellular response. An example is thrombin.

“Synthetic small molecule, “synthetic small molecule ligand,” “synthetic ligand”, “synthetic agonist”, “exogenous agonist”, exogenous ligand” and the like are used interchangeably herein to mean any compound made exogenously by natural or chemical means that can bind within the transmembrane domains of a G protein-coupled receptor or modified G protein-coupled receptor or modified PAR (i.e., DREADD) and facilitate activation of the receptor and concomitant activation of a desired family of G proteins.

As used herein the term “binding” can be used interchangeably with the terms “receptor-ligand binding” or “ligand binding,” to mean physical interaction between a receptor (e.g., a G protein-coupled receptor or a modified receptor) and a ligand (e.g., a natural ligand, (e.g., peptide ligand) or synthetic ligand (e.g., synthetic small molecule ligand)). Ligand binding can be measured by a variety of methods known in the art (e.g., detection of association with a radioactively labeled ligand).

In some aspects, the modified receptor can be a modified G-protein coupled receptor (GPCR). In some aspects, the modified GPCR can be a Gq, a Gi, a Gs or a G₁₂/G₁₃ receptor.

“G protein-coupled receptor” as used herein refers to a receptor that, upon binding of its natural ligand and activation of the receptor, transduces a G protein-mediated signal(s) that results in a cellular response. G protein-coupled receptors form a large family of evolutionarily related proteins. Proteins that are members of the G protein-coupled receptor family are generally composed of seven putative transmembrane domains. G protein coupled receptors were also known in the art as “seven transmembrane segment (7TM) receptors” and as “heptahelical receptors”. GPCRs detect molecules outside the cell and activate internal signal transduction pathways and, ultimately, cellular responses. GPCRs interact with a complex of heterotrimeric guanine nucleotide-binding proteins (G-proteins) and thus regulate a wide variety of intracellular signaling pathways including ion channels. For example, when a ligand binds to the GPCR it causes a conformational change in the GPCR, which allows it to act as a guanine nucleotide exchange factor (GEF). The GPCR can then activate an associated G protein by exchanging the GDP bound to the G protein for a GTP. The G protein's a subunit, together with the bound GTP, can then dissociate from the β and γ subunits to further affect intracellular signaling proteins or target functional proteins directly depending on the a subunit type (Gαs, Gαi/o, Gαq/11, Gα12/13). As used herein, a “G protein-coupled cellular response” or “GPCR cellular response” means a cellular response or signaling pathway that occurs upon ligand binding by a GPCR. Such GPCR cellular responses relevant to the present disclosure are those which trigger the activation of one or more platelets which in turn induces the release of one or more biomolecules and/or therapeutic agents. The type of response whether it is an inhibitory response or excitatory response will depend on biomolecule(s) and/or therapeutic agent released and the type of GPCR activated.

The term “signaling” as used herein can mean the generation of a biochemical or physiological response as a result of ligand binding (e.g., as a result of synthetic or exogenous ligand binding to a modified receptor).

The terms “receptor activation,” “DREADD activation,” “modified receptor activation” “GPCR activation” and “PAR activation” can be used interchangeably herein to mean binding of a ligand (e.g., a natural or synthetic ligand) to a receptor in a manner that elicits G protein-mediated signaling, and a physiological or biochemical response associated with G protein-mediated signaling. Activation can be measured by measuring a biological signal associated with G protein-related signals.

“Targeted cellular activation” and “target cell activation” can be used interchangeably herein to mean DREADD-mediated activation or receptor-mediated activation of a specific G protein-mediated physiological response in a target cell (e.g., an engineered platelet), wherein DREADD-mediated activation or receptor-mediated activation occurs by binding of an endogenous ligand molecule to the DREADD or modified receptor. As used herein, cellular activation can includes inducing the release of one or more biomolecules and/or therapeutic agents that in turn can elicit an inhibitory response or an excitatory response.

The compositions and methods described herein can affect or elicit G protein-mediated cellular response of a eukaryotic cell (e.g., a platelet). The platelet is a cell that has been engineered using a genetic circuit comprising a sequence capable of encoding the modified GPCR.

In some aspects, the modified receptor can be a modified protease-activated receptor (PAR). In some aspects, the modified PAR can be PAR1, PAR2, PAR3 or PAR4. PARs are a subfamily of related G protein-coupled receptors that are activated by cleavage of part of their extracellular domain. PARS are highly expressed in platelets. Most of the PAR family act through the actions of G-proteins i (cAMP inhibitory), 12/13 (Rho and Ras activation) and q (calcium signaling) to cause cellular actions.

Disclosed herein is a genetic circuit comprising one or more megakaryocyte differentiation genes. Also disclosed herein is a genetic circuit comprising one or more megakaryocyte differentiation genes and a promoter. Further disclosed herein is a nucleic construct comprising a first genetic circuit comprising a tissue-specific promoter operatively linked to a sequence capable of encoding a modified receptor; and further comprising a second genetic circuit, wherein the second genetic circuit comprises one or more megakaryocyte differentiation genes. In some aspects, the tissue-specific promoter of the first genetic circuit can be regulatable. In some aspects, the second genetic circuit can comprise a promoter. In some aspects, the second genetic circuit comprises a promoter, wherein the promoter is operatively linked to the one more megakaryocyte differentiation genes. In some aspects, the promoter of the second genetic circuit can be regulatable. In some aspects, the promoter of the second genetic circuit can be constitutively active. In some aspects, the tissue-specific promoter of the first genetic circuit can be operatively linked to the one or more megakaryocyte differentiation genes. In some aspects, the one or more megakaryocyte differentiation genes can be HoxB4, GATA-1, c-MYC, BMI1, BCL-XL, PLK-1 or a combination thereof. In some aspects, the one or more megakaryocyte differentiation genes can be HoxB4 or GATA-1 or any other differentiation genes that can direct differentiation of a hemopoietic stem cell to a megakaryocyte. In some aspects, the one or more megakaryocyte differentiation genes can be HoxB4 and GATA-1. In some aspects, the GATA-1 can comprise an auxin protein degradation tag. In some aspects, the one or more megakaryocyte differentiation genes can be c-MYC, BMI1, BCL-XL. In some aspects, the one or more megakaryocyte differentiation genes can be PLK-1 and/or any other differentiation genes that can direct differentiation of a pluripotent stem cell to a megakaryocyte. A differentiation gene(s) can be a gene that facilitates the process by which a less specialized cell becomes a more specialized cell type. Gene expression can regulate cell differentiation. A gene or a combination of genes that are turned on (expressed) or turned off (repressed) can dictate cellular morphology and function.

In some aspects, the first genetic circuit disclosed herein can further comprise a gene of interest. In some aspects, the second genetic circuit disclosed herein can further comprise a gene of interest. In some aspects, the gene of interest can be a therapeutic agent. A therapeutic agent can be an enzyme, a hormone, a polypeptide, an antibody, a drug, a chemotherapeutic agent, a toxin, or an oligonucleotides.

Disclosed herein is a genetic circuit comprising a gene of interest. Also, disclosed herein is a third genetic circuit, wherein the third genetic circuit comprises a gene of interest. Further disclosed herein is a nucleic construct comprising a first genetic circuit comprising a tissue-specific promoter operatively linked to a sequence capable of encoding a modified receptor; and further comprising a second genetic circuit, wherein the second genetic circuit comprises one or more megakaryocyte differentiation genes; and further comprising a third genetic circuit, wherein the third genetic circuit comprises a gene of interest. In some aspects, the second genetic circuit comprises a promoter, wherein the promoter is operatively linked to the one more megakaryocyte differentiation genes. In some aspects, the third genetic circuit comprises a promoter operatively linked to a gene of interest. In some aspects the promoter of the third genetic circuit can be regulatable. In some aspects, the gene of interest can be a therapeutic agent. A therapeutic agent can be an enzyme, a hormone, a polypeptide, an antibody, a drug, a chemotherapeutic agent, a toxin, or an oligonucleotides.

In some aspects, any of the genetic circuits described herein, can further comprise one or more recombination sites. In some aspects, the one or more recombination sites can be loxP, attP, Bxb1 or a combination thereof. In some aspects, the attP, loxP, or Bxb1 sites can be inserted at a Rosa26 locus. In some aspects, any of the genetic circuits described herein, can further comprise one or more repressor proteins. In some aspects, the one or more repressor proteins can be LacI, TetR, QS or a combination thereof. In some aspects, the one or more repressor proteins can be regulatable. In some aspects, any of the promoters in any of the genetic circuits described herein, can comprise one or more operator sites. In some aspects, in any of the genetic circuits disclosed herein, one or more of the genes described herein can be regulatable, constitutively active or a combination thereof. In some aspects, any of the genetic circuits disclosed herein can further comprise one or more recombinases. In some aspects, the one or more recombinases can be Cre, phiC31 integrase or Bxb1. In some aspects, the one or more recombinases can be regulatable.

Pluripotent stem cells. Disclosed herein are pluripotent stem cells. Disclosed herein are pluripotent stem cells comprising any of the nucleic acid constructs disclosed herein. Disclosed herein are pluripotent stem cells comprising any of the genetic circuits disclosed herein. In some aspects, the pluripotent stem cell can be a hematopoietic progenitor stem cell, an embryonic stem cell or an induced pluripotent stem cell (iPSC). In some aspects, the pluripotent stem cells are derived from cord blood or bone marrow. In some aspects, the iPSC can be derived from blood cells. In some aspects, the pluripotent stem cells can be human pluripotent stem cells.

Megakaryocytes. Disclosed herein are megakaryocytes. Disclosed herein are megakaryocytes at any development stage. Disclosed herein are megakaryocytes comprising any of the nucleic acid constructs described herein. Disclosed herein are megakaryocytes comprising any of the genetic constructs described herein. Disclosed herein are megakaryocytes comprising a nucleic acid construct, wherein the nucleic acid construct comprises a first genetic circuit comprising a tissue-specific promoter operatively linked to a sequence capable of encoding a modified receptor. In some aspects, the tissue-specific promoter can be a megakaryocyte-specific promoter. In some aspects, the megakaryocyte-specific promoter can be CXCL4, GPIIb, or PTPRC. In some aspects, the modified receptor can be a modified G-protein coupled receptor (GPCR) or a modified protease-activated receptor (PAR). In some aspects, the modified GPCR can be a Gq, a Gi, a Gs or a G₁₂/G₁₃ GPCR. In some aspects, the modified PAR can be PAR1, PAR2, PAR3 or PAR4. In some aspects, the megakaryocytes disclosed herein can further comprise a second genetic circuit. In some aspects, the second genetic circuit can comprise one or more megakaryocyte differentiation genes. In some aspects, the one or more megakaryocyte differentiation genes can be HoxB4, GATA1, c-MYC, BMI1, BCL-XL, PLK-1 or a combination thereof. In some aspects, the one or more megakaryocyte differentiation genes can be HoxB4 or GATA1 or any other differentiation genes that can direct differentiation of a hemopoietic stem cell to a megakaryocyte. In some aspects, the one or more megakaryocyte differentiation genes can be HoxB4 and GATA1. In some aspects, the one or more megakaryocyte differentiation genes can be c-MYC, BMI1, BCL-XL. In some aspects, the one or more megakaryocyte differentiation genes can be PLK-1 and/or any other differentiation genes that can direct differentiation of a hemopoietic stem cell to a megakaryocyte. In some aspects, the megakaryocyte comprising a first genetic circuit and/or a second genetic circuit can comprise an additional genetic circuit. In some aspects, the additional genetic circuit can comprise a gene of interest. In some aspects, the additional genetic circuit can be a second or a third genetic circuit. In some aspects, the gene of interest can be a therapeutic agent. A therapeutic agent can be an enzyme, a hormone, a polypeptide, an antibody, a drug, a chemotherapeutic agent, a toxin, or an oligonucleotides.

Also disclosed herein are engineered megakaryocytes comprising a modified receptor. In some aspects, the modified receptor can be a modified G-protein coupled receptor (GPCR) or a modified protease-activated receptor (PAR). In some aspects, the modified GPCR can be a Gq, a Gi, a Gs or a G₁₂/G₁₃ GPCR. In some aspects, the modified PAR can be PAR1, PAR2, PAR3 or PAR4. In some aspects, the any of the engineered megakaryocytes disclosed herein can further comprise a therapeutic agent.

Platelets. Disclosed herein are platelets. Disclosed herein are engineered platelets. Disclosed herein are engineered platelet comprising a modified receptor. In some aspects, the modified receptor can be a modified G-protein coupled receptor (GPCR) or a modified protease-activated receptor (PAR). In some aspects, the modified GPCR can be a Gq, a Gi, a Gs or a G₁₂/G₁₃ GPCR. In some aspects, the modified PAR can be PAR1, PAR2, PAR3 or PAR4. In some aspects, the any of the engineered platelets disclosed herein can further comprise a therapeutic agent.

Methods of Producing Platelets or Populations of Platelets

Disclosed herein are method of producing platelets or a population of platelets. Disclosed herein are methods of producing platelets or a population of platelets comprising a modified receptor. In some aspects, the methods can comprise: a) providing pluripotent stem cells comprising any of the nucleic acid constructs disclosed herein; b) culturing the pluripotent stem cells in a media under conditions to permit the expansion of the pluripotent stem cells to megakaryocytes; and c) differentiating the megakaryocytes into platelets; wherein the platelets or the population of platelets comprise the modified receptor. In some aspects, the pluripotent stem cell can be a hematopoietic progenitor stem cell, an embryonic stem cell or an induced pluripotent stem cell (iPSC). In some aspects, the pluripotent stem cell can be derived from cord blood or bone marrow. In some aspects, the iPSC can be derived from blood cells. In some aspects, the pluripotent stem cell can be a human pluripotent stem cell. In some aspects, the media can comprise one or more modulators. In some aspects, the media modulators can be used to direct differentiation of a stem cell to a specific cell type. In some aspects, the one or more media modulators can facilitate the differentiation of a pluripotent stem cell to a megakaryocyte. In some aspects, the one or more media modulators can facilitate the differentiation of a megakaryocyte to a platelet. In some aspects, the one or more media modulators can facilitate the differentiation of a pluripotent stem cell to a platelet. In some aspects, the one or more media modulators can be isopropyl β-D-1-thiogalactopyranoside (IPTG), tetracycline, doxacycline, quinic acid, or auxin. In some aspects, the modified receptor can be a modified G-protein coupled receptor (GPCR) or a modified protease-activated receptor (PAR). In some aspects, the modified GPCR can be a Gq, a Gi, a Gs or a G₁₂/G₁₃ GPCR. In some aspects, the modified PAR can be PAR1, PAR2, PAR3 or PAR4. In any of the methods disclosed herein, the platelets or the population of platelets further comprise a therapeutic agent. In some aspects, the pluripotent stem cells can comprise a nucleic acid construct comprising: a genetic circuit comprising a tissue-specific promoter operatively linked to a sequence capable of encoding a modified receptor; or wherein the pluripotent stem cells comprise a nucleic acid construct comprising a first genetic circuit comprising a tissue-specific promoter operatively linked to a sequence capable of encoding a modified receptor; and a second genetic circuit, wherein the second genetic circuit comprises one or more megakaryocyte differentiation genes; and wherein the first genetic circuit or the second genetic circuit further comprises a gene of interest. In some aspects, the method can further comprise isolating or purifying the platelets or population of platelets.

Also disclosed herein are methods of producing red blood cells and platelets. The method can comprise the following steps. The method can include step a): providing a genetically engineered feeder cell. The feeder cell can include one or more genetic circuits. The one or more genetic circuits can include one or more genes of interest; and one or more promoters. The method can include step b): providing a genetically engineered fed cell. The fed cell can include one or more genetic circuits. The one or more genetic circuits can include one or more genes of interest; and one or more promoters. The one or more genes of interest can be different than the one or more genes of interest in a). The method can further include step c): culturing the genetically engineered feeder cell in a) with the genetically engineered fed cell in b). The culturing step can take place in a media under conditions that permit the genetically engineered fed cells to differentiate into red blood cells or platelets. The one or more of the genetically engineered fed cells as disclose herein can differentiate into red blood cells or platelets.

In some aspects, the one or more genetic circuits in method step a) disclosed herein can be regulatable. In some aspects, the one or more genetic circuits can be regulated by one or more genes of interest of the genetic circuit in the genetically engineered fed cell. In some aspects, the one or more genetic circuits as disclosed herein can be regulated by the one or more genes of interest of the genetic circuit in the genetically engineered feeder cell.

In some aspects, the one or more genetic circuits as disclosed herein an in step a) can be regulated by one or more promoters. In some aspects, the one or more genetic circuits in step a) can further include one or more recombinases. In some aspects, the one or more recombinases can be, for example Cre or phiC31 integrase or Bxb1 integrase. In some aspects, the one or more recombinases can be regulatable. In some aspects, the one or more genetic circuits as disclosed herein and in a) can further include one or more recombination sites. In some aspects, the one or more recombination sites can be loxP, attP or Bxb1. In some aspects, the attP sites can be inserted at Rosa26 locus and/or in chromosome 11. As used herein, the term “promoter” refers to regulatory elements, promoters, promoter enhancers, internal ribosomal entry sites (IRES) and other elements that are capable of controlling expression (e.g., transcription termination signals, including but not limited to polyadenylation signals and poly-U sequences). Promoters can direct constitutive expression. Promoters can also direct expression in a temporal-dependent manner including but not limited to cell-cycle dependent or developmental stage-dependent. Examples of promoters include but are not limited to WPRE, CMV enhancers, and SV40 enhancers. Specific gene specific promoters can be used. Such promoters allow cell specific expression or expression tied to specific pathways. Any promoter that is active in mammalian cells can be used. In some aspects, the promoter is an inducible promoter including, but not limited to, Tet-on and Tet-off systems. Such inducible promoters can be used to control the timing of the desired expression. In some aspects, the promoter can be an inducible promoter. Examples of inducible promoters include but are not limited to tetracycline inducible system (tet); heat shock promoters and IPTG activated promoters. In some aspects, promoters are bidirectional.

The promoter and/or enhancer can be specifically activated either by light or specific chemical events which trigger their function. Systems can be regulated by reagents such as tetracycline and dexamethasone.

In some aspects, the genetic circuits as disclosed herein can comprise a promoter, for example but not limited to, enhancers, 5′ untranslated regions (5′UTR), 3′ untranslated regions (3′UTR), and repressor sequences; constitutive promoters, inducible promoter; tissue specific promoter, cell-specific promoter or variants thereof. Examples of tissue-specific promoters include, but are not limited to, albumin, lymphoid specific promoters, T-cell promoters, neurofilament promoter, pancreas specific promoters, milk whey promoter; hox promoters, a-fetoprotein promoter, human LIMK2 gene promoters, FAB promoter, insulin gene promoter, transphyretin, alpha.1-antitrypsin, plasminogen activator inhibitor type 1 (PAI-1), apolipoprotein myelin basic protein (MBP) gene, GFAP promoter, OPSIN promoter, NSE, Her2, erb2, and fragments and derivatives thereof. Examples of other promoters include, but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and variants thereof.

The one or more genetic circuits disclosed herein and in step a) can further include one or more repressor proteins. In some aspects, the one or more repressor proteins can be LacI, TetR, and/or QS. In some aspects, the one or more repressor proteins disclosed herein can be regulatable.

The media can further include one or more modulators. In some aspects, the one or more modulators can modulate (e.g., repress or activate) the genetic circuits of a) or b) as disclosed herein. In some aspects, the one or more genetic circuits disclosed herein an in step a) can be regulated by one or more media modulators. In some aspects, the one or more media modulators can be isopropyl β-D-1-thiogalactopyranoside (IPTG), tetracycline, doxacycline, quinic acid, or auxin.

The method disclosed herein can also include one or more genetic circuits in step a) that are non-regulatable. In some aspects, the one or more promoters of the genetic circuits as disclosed herein and in step a) can be constitutively expressed. In some aspects, the one or more promoters of the genetic circuits disclosed herein and in step a) can be CMV, RSV and/or U6, beta actin, and/or elongation factor promoters. In some aspects, the one or more promoters can include one or more operator sites (e.g., tet). Such operator sites can allow for one or more repressor proteins to bind.

The method disclosed herein can also include one or more genes of interest of the genetic circuits in step a). In some aspects, the one or more genes of interest of the genetic circuits disclosed herein can be erythropoietin, thrombopoietin, and/or IL1-α. In some aspects, the one or more genes of interest of the genetic circuits disclosed herein and in step a) can be constitutively expressed.

The method disclosed herein can include a genetically engineered feeder cell. In some aspects, the genetically engineered feeder cell can be derived from an embryonic stem cell or a mouse embryonic stem cell. In some aspects, the genetically engineered feeder cell can be an osteoblast. In some aspects, the osteoblast can be an OP-9 stromal cell. In some aspects, the osteoblast can be from cord blood or bone marrow. In some aspects, the genetically engineered feeder cell can be derived from an immortalized cell line. In some aspects, the genetically engineered feeder cell can support undifferentiated hematopoietic stem cell (HSC) growth. In some aspects, the genetically engineered feeder cell is capable of being genetically engineered.

The method disclosed herein can include a non-genetically engineered feeder cell. In some aspects, the feeder cell can be derived from an embryonic stem cell or a mouse embryonic stem cell. In some aspects, the feeder cell can be an osteoblast. In some aspects, the osteoblast can be an OP-9 stromal cell. In some aspects, the osteoblast can be from cord blood or bone marrow. In some aspects, the feeder cell can be derived from an immortalized cell line. In some aspects, the feeder cell can support undifferentiated hematopoietic stem cell (HSC) growth.

The methods disclosed herein can use a variety of cells. Examples of cells include but are not limited to stem cells, such as embryonic stem cells.

The method disclosed herein can include one or more genetic circuits as described herein and in b) that can be regulatable. In some aspects, the one or more genetic circuits in b) can be regulated by one or more genes of interest of the genetic circuit in the genetically engineered fed cell. In some aspects, the one or more genetic circuits in b) can be regulated by one or more genes of interest of the genetic circuit in the genetically engineered feeder cell. In some aspects, one or more genetic circuits in b) can further comprise one or more recombinases. In some aspects, one or more recombinases can be Cre or phiC31 integrase or Bxb1 integrase. In some aspects, one or more recombinases can be regulatable.

The method disclosed herein can include one or more genetic circuits as described herein and in step b) that further comprise one or more recombination sites. In some aspects, one or more recombination sites can be loxP, attP or Bxb1. In some aspects, the attP, loxP, or Bxb1 sites can be inserted at Rosa26 locus. In some aspects, the one or more genetic circuits disclosed herein and in step b) can be regulated by one or more promoters. In some aspects, one or more genetic circuits disclosed herein and in step b) can further comprise one or more repressor proteins. In some aspects, one or more repressor proteins can be LacI, TetR, or QS. In some aspects, one or more repressor proteins can be regulatable.

In some aspects, one or more genetic circuits disclosed herein and in step b) can be regulated by one or more media modulators. In some aspects, one or more modulators can be isopropyl β-D-1-thiogalactopyranoside (IPTG), tetracycline, doxacycline, quinic acid, or auxin. Such media modulators or agents are well known in the art.

The method disclosed herein can include one or more genetic circuits described herein and in step b) that can be non-regulatable. In some aspects, one or more promoters of the genetic circuits disclosed herein and in step b) can be constitutively active. In some aspects, one or more promoters of the genetic circuits in step b) can be CMV, RSV U6, beta actin, and/or elongation factor promoters. In some aspects, one or more promoters (e.g., CMV, RSV and/or U6) can comprise one or more operator sites. In some aspects, the operator sites can allow for repressor proteins to bind.

In some aspects, one or more genes of interest of the genetic circuits disclosed herein and in step b) can be HoxB4 and/or GATA-1. In some aspects, one or more genes of interest of the genetic circuits disclosed herein and in step b) can be constitutively expressed. In some aspects, GATA-1 comprises an auxin protein degradation tag.

In some aspects, the genetically engineered fed cells described herein can be hematopoietic progenitor stem cells. In some aspects, the hematopoietic stem cell can be derived from cord blood, bone marrow, iPS cell, or ES cell. In some aspects, the genetically engineered fed cell can be capable of producing progenitor cells of platelets and red blood cells. In some aspects, the progenitor cells can be capable of producing platelets and red blood cells. In some aspects, the progenitor cells can comprise one or more of the genetic circuits disclosed herein. In some aspects, the progenitor cells comprise one or more of the genetic circuits disclosed herein that can regulate the expression of any of the one or more genes of interest. In some aspects, one or more genes of interest can be HoxB4 and/or GATA-1. In some aspects, the genetic circuits described herein also can comprise one or more repressor proteins (e.g., LacI, TetR or QS) and can be controlled by one or more medial modulators (e.g., isopropyl β-D-1-thiogalactopyranoside (IPTG), tetracycline, doxacycline, quinic acid, or auxin).

The gene of interest can be any gene. It can be endogenous or introduced. The terms “target,” “target gene,” and “target nucleotide sequence” can be used interchangeably and refers to the gene of interest. For example, a target gene is a gene of known function or is a gene whose function is unknown, but whose total or partial nucleotide sequence is known. Alternatively, the function of a target gene and its nucleotide sequence are both unknown. A target gene can be a native gene of the eukaryotic cell or can be a heterologous gene which has previously been introduced into the eukaryotic cell or a parent cell of said eukaryotic cell, for example by genetic transformation. A heterologous target gene can be stably integrated in the genome of the eukaryotic cell or is present in the eukaryotic cell as an extrachromosomal molecule, e.g., as an autonomously replicating extrachromosomal molecule. A target gene can include polynucleotides comprising a region that encodes a polypeptide or polynucleotide region that regulates replication, transcription, translation, or other process important in expression of the target protein; or a polynucleotide comprising a region that encodes the target polypeptide and a region that regulates expression of the target polypeptide; or non-coding regions such as the 5′ or 3′ UTR or introns. A target gene may refer to, for example, an mRNA molecule produced by transcription a gene of interest.

The design or construction of the genetic circuits disclosed herein can be carried out in a modular fashion, allowing for the regulation of any gene, including heterologous and other recombinant genes. In some aspects, the parts or modules can be genetic activators, genetic repressors, recombinases, genome editing, and synthetic transcription factors. In some aspects, the genetic circuit described herein can comprise one or more modules.

Vectors can be introduced in a prokaryote, amplified and then the amplified vector can be introduced into a eukaryotic cell. The vector can also be introduced in a prokaryote, amplified and serve as an intermediate vector to produce a vector that can be introduced into a eukaryotic cell (e.g., amplifying a plasmid as part of a viral vector packaging system). A prokaryote can be used to amplify copies of a vector and express one or more nucleic acids to provide a source of one or more proteins for delivery to a host cell or host organism. Expression of proteins in prokaryotes is often carried out in Escherichia coli with vectors containing constitutive or inducible promoters directing the expression of either fusion or non-fusion proteins. Vectors can also be a yeast expression vector (e.g., Saccharomyces cerevisiae).

In some aspects, the vector is capable of driving expression of one or more sequences in mammalian cells using a mammalian expression vector. Examples of mammalian expression vectors include but are not limited to pCDM8 and pMT2PC. In mammalian cells, regulatory elements control the expression of the vector. Examples of promoters are those derived from polyoma, adenovirus 2, cytomegalovirus, simian virus 40, and others disclosed herein and known in the art.

In some aspects, the methods disclosed herein can include conditions in step c) that permit the expression of one or more genes of interest in steps a) or b). In some aspects, osteoblasts can be contacted, exposed to or treated with mitomycin-C. The osteoblasts can be washed before the stem cells are added. The osteoblasts can be washed to remove the mitomycin-C. Generally, the osteoblasts can be prepared accordingly to standard protocol that is known to one of ordinary skill in the art. The osteoblasts, for example, can be treated with mitomycin-C prior to or just before growing additional cells on top of the feeder cells.

In some aspects, the medium that can be used in the methods disclosed herein can comprise one or more components or modulators (e.g., media modulators). The one or more components or modulators can lead to the formation of platelet and/or red blood cell progenitor stem cells. In some aspects, one or more components or modulators described herein can be isopropyl β-D-1-thiogalactopyranoside (IPTG), tetracycline, doxacycline, quinic acid, or auxin. In some aspects, the progenitor stem cells can produce platelet and/or red blood cell precursor cells. In some aspects, the progenitor stem cells can express one or more self-identifying cell surface markers. In some aspects, the progenitor stem cells can express GATA-1 and/or HoxB4. In some aspects, the expression of one or more cell surface markers can be produced by the genetic circuit disclosed herein. In some aspects, the one or more cell surface markers can be self-identifying. In some aspects, one or more cell surface markers can be CD13, CD34, CD41a, and CD43.

In some aspects, the platelets and/or red blood cells produced by the method described herein can express one or more cell surface markers. In some aspects, one or more cell surface markers can be CD41a and CD42b.

In some aspects, the method disclosed herein can further comprise step d): isolating or purifying the platelets or red blood cells.

Methods of Treating

Disclosed herein are methods of treating a human patient. In some aspects, the methods can comprise: a) administering one or more of the platelets or population of platelets described herein to a human patient; and b) administering an exogenous agonist to the human patient; wherein the presence of the exogenous agonist activates the modified receptor. In some aspects, the activation of the modified receptor can induce the release of the therapeutic agent from any of the platelets disclosed herein or one or more endogenous molecules from any of the platelets disclosed herein. In some aspects, any of the platelets or the population of platelets disclosed herein can be administered via intravenous injection or transfusion. In some aspects, the one or more platelets or the population of platelets disclosed herein can be administered via intravenous injection or transfusion. In some aspects, the exogenous ligand or agonist can be administered via intracranial, instraspinal, intramuscular, or intravenous injection or orally. In some aspects, the exogenous ligand or agonist can be selective for or specific to the modified receptor present on the engineered platelet. In some aspects, the human patient has been identified as being in need of treatment before the administration step. In some aspects, the human patient can have a disease or a disorder.

In some aspects, any of the platelets described herein can be administered via a transfusion to a subject or patient in any amount wherein the amount is sufficient to elicit a therapeutic response. In some aspects, the platelet concentration can be at least 1,000 μl, 5,000 μl, or 10,000 μl or within a range of 1,000 μl to 5,000 μl, 5,000 μl to 10,000 μl or any amount in between.

Administration regimen” or “support regimen” can refer to a schedule of platelet administration comprising amounts and types of platelets or other cells administered in accordance with a determined mode (such as continuous or intermittent) at a specific rate wherein mode or rate may vary with time. “Optimized administration regimen” or “optimized support regimen” refers to an administration or support regimen that is optimized by selecting platelets in accordance with a molecular attribute of the intended recipient.

Also disclosed are methods of delivering a therapeutic agent or one or more endogenous biomolecules to one or more cells. In some aspects, the methods comprise: contacting the one or more cells with one or more of the platelets disclosed herein. In some aspects, the contacting step can be done in the presence of an exogenous agonist. In some aspects, the presence of the exogenous agonist can activate the modified receptor thereby releasing the therapeutic agent or the one or more endogenous biomolecules to the one or more cells. In some aspects, the contacting step can be in vivo via intracranial, instraspinal, intramuscular, or intravenous injection. In some aspects, the one or more platelets or the population of platelets can be administered via intravenous injection or transfusion. In some aspects, the one or more platelets or the population of platelets can be administered to a subject or patient in need thereof. In some aspects, the exogenous agonist can be administered to a subject or patient in need thereof. In some aspects, the exogenous agonist can be administered before, during or after the contacting step. In some aspects, the exogenous agonist can be administered via intracranial, instraspinal, intramuscular, or intravenous injection or orally. In some aspects, the exogenous ligand or agonist can be selective for or specific to the modified receptor present on the engineered platelet. In some aspects, the human patient has been identified as being in need of treatment before the administration step. In some aspects, the human patient can have a disease or a disorder.

Diseases and disorders. In some aspects, the disease can be a lysosomal storage disease. In some aspects, the disease can be cancer. In some aspects, the disease can be diabetes.

In some aspects, the disease can be an autoimmune disease or disorder. In some aspects, the autoimmune disease or disorder can affect an organ. In some aspects, the affected organ can be heart, kidney, liver, lung, or skin. In some aspects, the autoimmune disease or disorder can affect a gland. In some aspects, the gland can be the adrenal gland, multi-glandular, pancreas, thyroid gland, one or more reproductive organs, or salivary glands. In some aspects, the autoimmune disease or disorder can affect the digestive system. In some aspects, the autoimmune disease or disorder can affect the blood, connective tissue, be systemic and/or multi-organ, muscle, nervous system, eyes, ears, or vascular system. In some aspects, the autoimmune disease or disorder can be rheumatoid arthritis, systemic lupus erythematosus, inflammatory bowel disease (e.g., ulcerative colitis and Crohn's disease), type I diabetes mellitus, Guillian-Barre syndrome, chronic inflammatory demyelinating polyneuropathy, psoriasis, Grave's disease, Hashimoto's thyroiditis, Myasthenia gravis, multiple sclerosis, Addison's disease, Sjogren's syndrome, pernicious anemia, celiac disease, and vasculitis.

In some aspects, the disease can be a cancer. In some aspects, the cancer can be a primary or secondary tumor. In some aspects, the cancer has metastasized. In some aspects, the cancer can be a solid cancer or a blood cancer. The cancer can be any cancer. In some aspects, the cancer can anal cancer, bladder cancer, brain cancer, bone cancer, breast cancer, cervical cancer, colorectal cancer, endocrine cancer, esophageal cancer, eye cancer, gallbladder cancer, head and neck cancer, kidney cancer, leukemia, liver cancer, lymphoma, melanoma, oral or oropharyngeal cancer, osteosarcoma, parathyroid cancer, pancreatic cancer, penile cancer, pituitary gland cancer, prostate cancer, skin cancer, stomach cancer, testicular cancer, thyroid cancer, uterine cancer, vulvar cancer, ovarian cancer, lung cancer, or gastric cancer.

Agonists and Administration. Disclosed herein are modified receptors that can be activated by the presence of an exogenous agonist. The exogenous agonist (or ligand, or small molecule, the terms are used interchangeably herein) is one which can be delivered orally or parenterally (e.g., systemically administered). The ligand is exogenous in that it is generally absent from the body or area to be treated or targeted for release of one or more biomolecules or a therapeutic agent, or is present in sufficiently low basal concentrations that it does not activate the modified receptor. In some aspects, the ligand can be synthetic, i.e., not naturally occurring. In some aspects, ligand is one that possesses minimal or no biologic activity other than DREADD activation or modified receptor activation.

Any small molecule, generally a synthetic small molecule that can bind within the transmembrane domains of the DREADD or modified receptor and facilitate DREADD-mediated activation or modified receptor-mediated activation of a desired family of G proteins is suitable for use in the method of targeted activation of the platelets described herein. In contrast to the natural peptide ligands of G protein-coupled receptors which typically have molecular weights of 2000-6000 Da, in some aspects, small molecule ligands of G protein-coupled receptors will generally have molecular weights of 100-1000 Da.

Synthetic small molecules useful in the methods disclosed herein include synthetic small molecules generated by either a natural (e.g., isolated from a recombinant cell line) or chemical means (e.g., using organic or inorganic chemical processes).

Several synthetic small molecules that bind and activate native GPCRs are known in the art and can be useful in the methods disclosed herein. Additional synthetic small molecules suitable for use in the methods disclosed herein can be identified by screening candidate compounds for binding to native GPCRS or to DREADDs. For example, by using a cell line expressing (or transfected with) a modified receptor or a DREADD and exposing it to varying concentrations of a compound to be tested for modified receptor or DREADD binding. Modified receptor or DREADD binding can be detected exposure to the test compound, but not in the presence of a control compound that does not bind the modified receptor or DREADD and/or does not induce cellular activation.

In some aspects, the ligand can be clozapine-N-oxide (CNO), which is a metabolite of clozapine. In some aspects, the ligand can be perlapine, which binds to hM3Dq. Since the binding sites of hM3Dq and hM4Di are highly similar, it can likewise be expected to bind hM4Di.

Agonists and Dosage. The term “treatment,” as used herein in the context of treating a disease or disorder, can relate generally to treatment and therapy of a human subject or patient, in which some desired therapeutic effect is achieved, for example, the inhibition of the progress of the disease or disorder, and can include a reduction in the rate of progress, a halt in the rate of progress, regression of the disease or disorder, amelioration of the disease or disorder, and cure of the disease or disorder. Treatment as a prophylactic measure (i.e., prophylaxis, prevention) is also included.

In some aspects, the exogenous ligand can be delivered in a therapeutically-effective amount. In some aspects, the platelets, engineered platelets or the population of platelets can be delivered in a therapeutically-effective amount.

The term “therapeutically-effective amount” as used herein, refers to the amount of the modified receptor or exogenous ligand that is effective for producing some desired therapeutic effect, commensurate with a reasonable benefit/risk ratio, when administered in accordance with a desired treatment regimen.

Similarly, the term “prophylactically effective amount,” as used herein refers to the amount of the modified receptor or exogenous ligand that is effective for producing some desired prophylactic effect, commensurate with a reasonable benefit/risk ratio, when administered in accordance with a desired treatment regimen. “Prophylaxis” as used herein refers to a measure which is administered in advance of detection of a symptomatic condition, disease or disorder with the aim of preserving health by helping to delay, mitigate or avoid that particular condition, disease or disorder.

While it may possible for the exogenous ligand to be used (e.g., administered) alone, it is often preferable to present it as a composition or formulation e.g. with a pharmaceutically acceptable carrier or diluent.

In some aspects, the CNO can be administered via parenteral administration. In some aspects, the CNO can be administered via oral administration. In some aspects, the dosage of CNO administered can be between 0.1 mg/kg and 20 mg/kg. In some aspects, the dosage of CNO administered can be between 1 mg/kg and 5 mg/kg.

The term “pharmaceutically acceptable,” as used herein, relates to compounds, ingredients, materials, compositions, dosage forms, etc., which are, within the scope of sound medical judgment, suitable for use in contact with the tissues of the subject (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

In some aspects, the composition can be a pharmaceutical composition (e.g., formulation, preparation, medicament) comprising, or consisting essentially of, or consisting of as a sole active ingredient, a ligand as described herein, and a pharmaceutically acceptable carrier, diluent, or excipient.

In some aspects, the disclosed methods or compositions can be combined with other therapies, whether symptomatic or disease modifying.

The term “treatment” includes combination treatments and therapies, in which two or more treatments or therapies are combined, for example, sequentially or simultaneously. For example it may be beneficial to combine treatment with a compound as described herein with one or more other (e.g., 1, 2, 3, 4) agents or therapies. Appropriate examples of co-therapeutics are known to those skilled in the art based one the disclosure herein. Typically the co-therapeutic can be any known in the art which it is believed may give therapeutic effect in treating the diseases or disorders described herein, subject to the diagnosis of the individual being treated. The particular combination would be at the discretion of the physician who would also select dosages using his/her common general knowledge and dosing regimens known to a skilled practitioner.

The agents (e.g., engineered platelet comprising the modified receptor and exogenous ligand, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

Disclosed herein are methods of treating a patient. In some aspects, the patient can be in need of a platelet transfusion. In some aspects, the methods can comprise administering a therapeutically effective amount of the in vitro produced and optionally isolated platelets. The in vitro produced and optionally isolated platelets can be produced by any of the methods disclosed herein.

Disclosed herein are methods of producing platelets comprising a therapeutic agent, peptide, enzyme or bioactive molecule (biomolecule). In some aspects, the methods can comprise any of the methods disclosed herein to produce platelets harboring therapeutic proteins within them to be released in the body. In some aspects, the methods can comprise extrinsic and/or intrinsic regulation as described herein. In some aspects, the methods can also include engineering the platelets to comprise receptors capable of activating the platelets to trigger the release of, for example, enzymes upon binding to specific drugs and/or binding to tissue specific peptides.

Disclosed herein are methods of producing platelets comprising therapeutic agents, peptide, enzyme or bioactive molecule (biomolecules). In some aspects, the methods can comprise the steps: a) providing a genetically engineered osteoblast; b) providing a genetically engineered hematopoietic stem cell (HSC), wherein the HSC comprises one or more genetic circuits; wherein the one or more genetic circuits comprise one or more genes of interest, wherein the one or more genes of interest are different than the one or more genes of interest in a); and one or more promoters; c) culturing the genetically engineered osteoblast in a) with the genetically engineered HSC in b) in a media under conditions that permit the genetically engineered HSC to differentiate into platelet stem cells; and d) producing platelets comprising therapeutic agents, peptide, enzyme or bioactive molecule. In some aspects, the platelets that are produced can comprise an engineered receptor or a modified receptor. In some aspects, the genetically engineered HSC can be from a pluripotent stem cells or one of their progenitor stem cells. The progenitor stem cells are capable of producing the therapeutic agent, peptide, enzyme or bioactive molecule. The progenitor stem cells can be regulated intrinsically or extrinsically to produce or secrete the therapeutic agent, peptide, enzyme or bioactive molecule. The methods can also include engineering the platelets to comprise receptors capable of activating platelets to trigger the release of enzymes upon binding to specific drugs and/or binding to tissue specific peptides.

As used herein, the term “therapeutic agent” refers to a chemical compound, a protein, a peptide, a small molecule, an antibody, a gene, an enzyme or a cell.

In some aspects, the therapeutic proteins or agents as disclosed herein can be transcribed from genetic circuits in platelet progenitor stem cells, prior to the terminal differentiation into platelets. In some aspects, the therapeutic proteins or agents as disclosed herein can be transcribed from genetic circuits in megakaryocytes. These therapeutic proteins or agents can be present in the cytoplasm of progenitor cells and, therefore, be a part of the terminally differentiated platelets. The production of therapeutic proteins or agents can be transcribed from constitutively expressing promoters, and/or with inducible genetic circuits.

In some aspects, the method disclosed herein can further comprise the step: e) re-culturing the progenitor stem cells produced step c) in a media under conditions promoting the differentiation of the progenitor stem cells into platelets. In some aspects, the method disclosed herein can further comprise the step: f) collecting or isolating the platelets.

In some aspects, the methods disclosed herein can be carried out to produce therapeutic cells. Therapeutic cells can comprise one or more therapeutic agents, peptides, enzymes, genes or bioactive molecule. In some aspects, the therapeutic agent can be a small molecule, a gene, a peptide, an enzyme, a vaccine, or an antimicrobial.

In some aspects, the one or more genetic circuits in a) are regulatable. In some aspects, the one or more genetic circuits in a) can be regulated by the one or more genes of interest of the genetic circuit in the genetically engineered HSC. In some aspects, the one or more genetic circuits in a) can be regulated by one or more promoters. In some aspects, the one or more promoters of the genetic circuit in a) and b) can be CMV, RSV and/or U6. In some aspects, the one or more promoters (e.g., CMV, RSV and/or U6) can comprise an operator site (e.g., tet).

In some aspects, the one or more genetic circuits in a) can further comprise one or more recombinases. In some aspects, the one or more recombinases can be Cre, phiC31 integrase and/or Bxb1. In some aspects, the one or more recombinases can be regulatable. In some aspects, the one or more genetic circuits in a) can further comprise one or more recombination sites. In some aspects, the one or more recombination sites can be loxP or attP. In some aspects, the attP or any other recombinase recognition sites can be inserted at Rosa26 and/or chromosome 11 locus. In some aspects, the attP and any other integrase recognition cites can serve as the insertion site for the therapeutic agent.

In some aspects, the one or more genetic circuits in a) can further comprise one or more repressor proteins. In some aspects, the one or more repressor proteins can be LacI, TetR, and/or QS. In some aspects, the one or more repressor proteins can be regulatable.

In some aspects, the media disclosed herein can further comprise one or more components or modulators. In some aspects, the one or more genetic circuits in a) and b) can be regulated by one or more media modulators or components. In some aspects, the one or more media modulators or components can be isopropyl β-D-1-thiogalactopyranoside (IPTG), tetracycline, doxacycline, quinic acid, or auxin.

In some aspects, the one or more genes of interest of the genetic circuit in a) can be thrombopoietin. In some aspects, thrombopoietin can be constitutively expressed.

In some aspects, the one or more genetic circuits in b) can be regulatable. In some aspects, the one or more genetic circuits in b) can be regulated by the one or more genes of interest of the genetic circuit in the genetically engineered HSC. In some aspects, the one or more genetic circuits in b) can be regulated by one or more promoters. In some aspects, the one or more promoters of the genetic circuit in b) can be CMV, RSV and/or U6.

In some aspects, the one or more genetic circuits in b) can further comprise one or more recombinases. In some aspects, the one or more recombinases can be phiC31 integrase or Cre or Bxb1 integrase. In some aspects, the one or more recombinases can be regulatable. In some aspects, the one or more genetic circuits in b) can further comprise one or more recombination sites. In some aspects, the one or more recombination sites can be loxP, attP or Bxb1. In some aspects, the attP, loxP or Bxb1 sites can be inserted at Rosa26 locus. In some aspects, the one or more recombination sites can serve as the insertion site for the therapeutic agent.

As described herein, the recombinase sites in the genome, for example, attP, can be used to insert any of the genetic circuits disclosed herein into the genome via a ‘docking site.’ This docking site allows for the targeted and robust insertion of the genetic circuits disclosed herein into the genome that are known to be robust in achieving gene expression and can be resistant to epigenetic silencing.

The location of the therapeutic agent can be in the genome.

In some aspects, the one or more genetic circuits in b) can further comprise one or more repressor proteins. In some aspects, the one or more repressor proteins can be LacI, TetR, and/or QS. In some aspects, one or more repressor proteins can be regulatable.

In some aspects, the media disclosed herein can further comprise one or more media modulators. In some aspects, the one or more media modulators can be isopropyl β-D-1-thiogalactopyranoside (IPTG), tetracycline, doxacycline, quinic acid, or auxin.

In some aspects, the one or more genes of interest of the genetic circuit in b) can be GATA-1. In some aspects, GATA-1 can be constitutively expressed.

In some aspects, the platelet progenitor stem cells in step c) can express one or more cell surface markers. In some aspects, the platelet progenitor stem cells in step c) can express GATA-1. In some aspects, the one or more surface markers can be CD13, CD34, CD41a, and CD43. In some aspects, the platelets or red blood cells can express one or more cell surface markers. In some aspects, the one or more cell surface markers can be CD41a and CD42b.

Disclosed herein are methods of treating a patient in need of a therapeutic agent. The method can comprise administering a therapeutically effective amount of therapeutic platelets to the subject or patient. The method can comprise identifying a patient in need of treatment before the administration step. The method can comprise administering to the patient a therapeutically effective amount of the isolated platelets. In some aspects, the platelets comprise a therapeutic agent. The isolated platelets and red blood cells do not contain DNA. These cells express the proteins and peptides that they were engineered to express via the methods disclosed herein. These cells are anucleated.

Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of condition disorder or disease.

The platelets as well as the platelets and red blood cells comprising a therapeutic agent described herein can be administered to the subject (e.g., a human patient) in an amount sufficient to delay, reduce, or preferably prevent the onset of clinical disease. Accordingly, in some aspects, the patient is a human patient. In therapeutic applications, compositions are administered to a subject (e.g., a human patient) already with or diagnosed with a condition, disorder or disease in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a platelets as well as the platelets comprising a therapeutic agent described herein can be an amount that achieves a cure, but that outcome is only one among several that can be achieved. One or more of the symptoms can be less severe. Recovery can be accelerated in an individual who has been treated.

The therapeutically effective amount of one or more of the therapeutic agents present within the platelets described herein and used in the methods as disclosed herein applied to mammals (e.g., humans) can be determined by one of ordinary skill in the art with consideration of individual differences in age, weight, and other general conditions (as mentioned above).

The platelets including platelets comprising a therapeutic agent described herein can be formulated for administration by any of a variety of routes of administration.

The platelets including platelets comprising a therapeutic agent can be prepared for parenteral administration. Platelets prepared for parenteral administration includes those prepared for intravenous (or intra-arterial), intramuscular, subcutaneous, and intraperitoneal, administration.

### Examples

### Example 1: Engineer Pluripotent Stem Cells to Regulate the Intrinsic Cues for Enhanced Differentiation

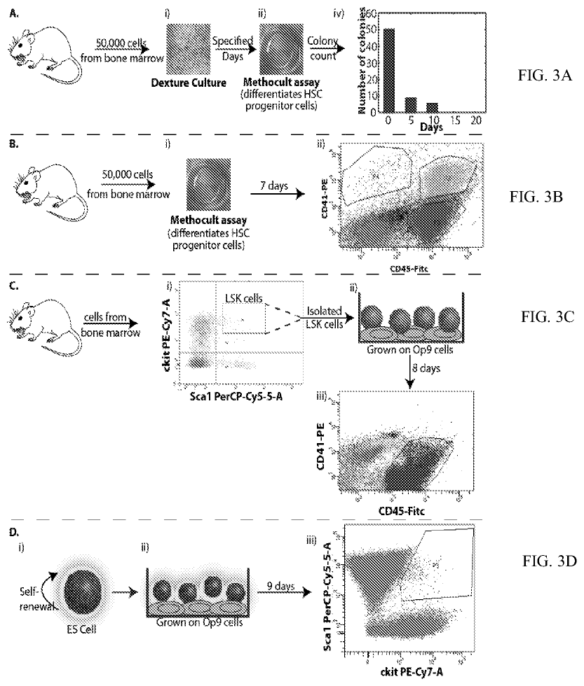

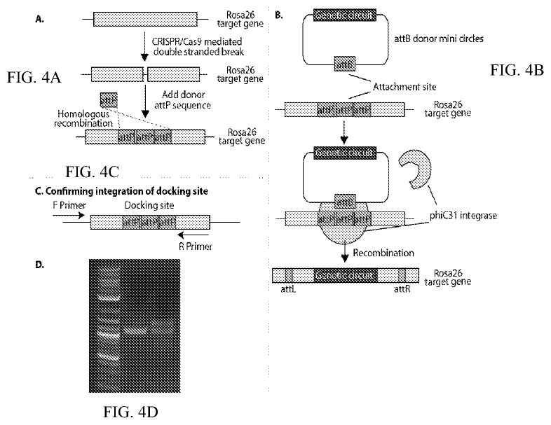

To determine whether mouse HSCs provide an adequate cell source for using the genetic tools, tools and methods were developed to load platelets, whole bone marrow was removed from mice and tested for long-term potential (FIG. 3A). Consistent with other reports, the number of lineage-committed HSC progenitors significantly decline over time²⁷⁻³² (FIG. 3B). Next, to determine whether isolated HSCs from the bone marrow could be differentiated into platelets in vitro, multipotent HSCs (LSK+ cells) were isolated from mouse bone marrow and differentiated them using standard differentiation conditions³³⁻³⁵ (FIG. 3C). From these studies, it was concluded that because HSCs have such a short lifetime in culture, genetically manipulating them directly for these purposes is not realistic. Next, the efficacy of differentiating embryonic stem cells (ES) into multipotent HSCs in vitro was tested and it was found that multipotent HSCs (LSK+) cells could be obtained within 9 days of culturing (FIG. 3D). Therefore, mouse embryonic stem (ES) will be genetically manipulated because these cells proliferate quickly, they renew their pluripotent cell population consistently, and they are easy to maintain in culture. These genetically manipulated ES cells will be differentiated into HSCs. Another advantage of this ES cell approach is that the engineered cells can be rapidly expanded and maintained longer than alternative cell lines such as primary HSCs, which rapidly reach senescence or spontaneously differentiate and therefore have a shorter functional lifetime. To accomplish this, a modular technology that utilizes CRISPR technology to insert a ‘landing pad’ or ‘docking site’ for genetic circuits was implemented. Conventional techniques involve the random insertion of genetic circuits into the genome, selecting stable clones in which the circuit is stably integrated, and testing the functionality of the circuit at that particular insertion site. These steps can be tedious and time consuming. Furthermore, the testing phase is significant because each clone may be different and positional affects (e.g., due to local enhancers, repressors, or epigenetic modifications) that may lead to the deregulation or misregulation of the circuit. To bypass these limitations, mouse embryonic stem (ES) cells have been engineered with ‘docking sites’ in the Rosa26 locus to allow for targeted and robust insertion of genetic circuits. The Rosa26 locus is widely used for achieving robust gene expression in mouse models and is resistant to epigenetic silencing³⁶. Using CRISPR/Cas9 technology, three attP sites have been added to the Rosa26 locus (FIG. 4), which allows unidirectional recombination at these sites to insert genetic circuits specifically at this locale using phiC31 integrase (FIG. 4B)³⁷. This allows a robust methodology for inserting any gene network into the genome of mouse ES cells. Furthermore, because ES cells are totipotent, these genetically modified cells can be differentiated into a range of different functional cell types based upon the disease or tissue of interest. For the purpose of platelet production and producing lysosomal enzymes in MKs, genetically altered ES cells will be differentiated into HSCs and the expression of lysosomal enzymes will be controlled at different stages throughout differentiation. Furthermore, developing this technology allows for any genetic background of cell type to be used and ensures immune compatibility with the various mouse models that are used. Finally, use of CRISPR technology will allow similar docking sites to be built in human cell lines.

### Example 2: Genetically Engineer Megakaryocytes to Create Platelets that Secrete Biomolecules

Platelets possess many characteristics that make them attractive candidates for in vivo delivery of natural and synthetic payloads: 1) they have extensive circulation range in the body, 2) they are a nucleated cells, 3) they are biocompatible, 4) their average lifespan in humans is ˜10 days, and 5) following activation, their protein granules serve as secretory vesicles, releasing components to the extracellular fluid. By using synthetic biology as disclosed herein, MKs can be programmed to express therapeutic levels of protein cargo to be targeted for platelet secretion. As a proof of concept, enhanced green fluorescence protein (EGFP), secreted alkaline phosphatase (SEAP), and luciferase will initially be expressed in MKs to determine the efficacy of using platelets as delivery vehicles for therapeutic payloads. This suite of reporter molecules has been selected because they can be used to assay different aspects of the cargo loading and delivery process. EGFP will be used to determine if soluble transgenic cargos are packaged into secretory granules, SEAP will be used to assay the extent of cargo release into the media of cells grown in vitro, and luciferase will be used to determine whether engineering platelets are enriched to sites of injury similar to endogenous platelets, to be used once these studies are moved to in vivo models.

Experiments and methodology. As a proof of concept to use platelets as delivery vehicles for therapeutic biomolecules, constitutively expressing reporter genes will be inserted into the attP site of ES cells (FIG. 4). These cells will be plated on OP9 stromal support cells and differentiated into MKs. Alternatively, human iPS cells can be used, and thus, these cells will not need to be plated on any support cell. The attP landing pad in ES cells will be used that were engineered to insert constitutively expressing reporter genes, differentiate these cells into MKs and platelets, then assay these cells for reporter expression to determine the location and function of these recombinantly made proteins and how they affect platelet function.

Express GFP in MKs and platelets: Like many potential bio-therapeutic molecules, GFP is a small, soluble protein that diffuses throughout the cytoplasm. MKs will be harvested for FACS analysis to confirm MK differentiation, and GFP expression level. The percent of GFP expressing MKs in the whole population will also be assessed. After determining the GFP expression level in MKs, MKs expressing GFP will be differentiated into platelets. FACs analysis will be done to confirm platelet differentiation and to quantify the GFP expression in these cells. In order to establish the sub-cellular distribution of GFP in platelets purified cells will be immunolabeled using antibodies against GFP.

Express SEAP in MKs and platelets: After differentiating HSCs to MKs on a layer of OP9 stromal cells, these cells will be harvested and SEAP secretion will be quantified in the media using established ELISA protocols³⁸. After determining SEAP secretion from MKs, the MKs expressing SEAP will differentiate into platelets and it will be determined whether platelets are capable of secreting biomolecules in vitro. To accomplish this, the engineered platelets will be characterized with non-engineered platelets by testing levels of SEAP in the culture media over multiple time points (three times a day for 10 days).

Express luciferase in MKs and platelets: To determine whether the engineered platelets are capable of responding to injury, luciferase will be expressed in MK cells and platelets, which will allow for live animal imaging. These experiments will serve as proof of concept for engineering platelets that are capable of expressing luciferase. After differentiating HSCs into MKs, luciferase activity will be quantified using a plate reader that is capable of bioluminescence.

Platelet characterization. Platelet cell differentiation can be identified by surface markers using flow cytometry. Degranulation and aggregation assessments will be made with respect to known activators von Willebrand Factor (vWF)³⁹, fibrinogen⁴⁰, collagen^{41,42}, and thrombin⁴³. Platelet degranulation will be determined by ELISA specific to serotonin and platelet derived factor 4 (PDF-4)⁴⁴. As a control, freshly isolated platelets from mice will be used for comparison. Platelet ability to aggregate in the presence of known activators will be determined using an aggregometer. Additionally, platelet degranulation by thrombin, which acts by enzymatically cleaving PAR receptors on platelets⁴³, will be determined by ELISA specific to serotonin and platelet derived factor 4 (PDF-4)⁴⁴.

In the event that GFP is not expressed in platelets, myristol-tagged GFP that has been shown to associate with the cell membrane⁴⁵ will be used. In this case, the GFP will associate with the MK membrane and is likely to become a part of the platelet membrane. Microscopy and flow cytometry will be done to observe and quantify GFP expression. In the event that SEAP or luciferase are not a part of the platelets, the reporter genes can be tagged with the amino acid sequence, LKNG (SEQ ID NO: 1), which has been demonstrated to be directly involved in the targeting and/or storage of the megakaryocytic proteins⁴⁶. To accomplish this, LKNG (SEQ ID NO: 1) can be fused to the reporter molecules in either the 5′ or 3′ UTR to be targeted for granule packaging in MKs.

### Example 3: Develop and Validate Directed Evolution Approaches for Engineering Novel Platelet Receptors

Platelets can become activated to secrete their bioactive molecules via G-protein coupled receptor (GPCR) signaling⁴⁷. GPCRs are a large family of versatile membrane proteins that have been the focus of many therapeutic targets because of their involvement in a range of normal and pathological diseases. To obtain precise spatiotemporal control of GPCR signaling in vivo, Designer Receptors Exclusively Activated by Designer Drugs (DREADDs) have been engineered to selectively, rapidly, reversibly, and dose-dependently control behaviors and physiological processes in the mammalian brain⁴⁸. These engineered receptors have been designed to have no endogenous ligand, no background activity in the absence of the ligand, and an otherwise pharmacologically inert compound exclusively and potently activates the GPCR by nanomolar concentrations of pharmacologically inert and metabolically stable small molecules⁴⁹. Since platelets use GPCRs as one of their means of activation, it is possible to engineer DREADDs on platelets as a strategy for spatially and temporally controlling the activation of these cells.

Experiments and Methodology. DREADDs have previously been engineered to enable non-invasive control of neuron signaling through the G_{q}, Gᵢ, and Gₛ G-protein coupled signaling pathways. To engineer receptors that are activated by a synthetic ligand and possess no biological activity, the GPCRs responsible for activating platelets will be modified to favor synthetic over endogenous substrate/ligand recognition. At the forefront of such modified GPCRs are the protease-activated receptors (PARs), which couple to G_{q}, G₁₂/G₁₃ and in some cases the Gᵢ family of G proteins. PARs are activated by thrombin, the most effective activator of platelets⁴⁷. Of the four PARs, PAR1 and PAR4 are present on human platelets, whereas mouse platelets express PAR3 and PAR4⁴⁷. For this reason, a directed molecular evolution approach will be taken to facilitate the creation of a family of PARs to be activated by the pharmacologically inert compound clozapine-N-oxide (CNO) but not by its native ligand thrombin. For these studies, CNO will be used as the synthetic ligand because the most widely used DREADDs today use CNO as their ligand and studies have shown that it is a pharmacologically inert molecule lacking affinity for innate receptors, and activates spatial and temporal control of GPCR signaling at nanomolar concentrations^{48,49}.

Directed molecular evolution is a technique for endowing a particular property to a protein by successive rounds of random mutation, screening, and then selection. Successfully evolving a protein to meet the desired criteria depends on several aspects of the experimental design including the biological diversity and size of the library to be screened, the quality of the screening assay, and the size of the functional jump from the template to the desired result. In the case of PAR receptors, which primarily engage the G_{q} signaling pathways, it is anticipated that a few to moderate functional steps will be carried out to evolve these receptors for CNO to activate signaling since the original DREADDs targeted receptors also signal via the G_{q} signaling pathways. In addition to designer drugs, it is also possible to evolve a receptor to respond to other drugs or to tissue specific peptides that would activate platelets and thus release their biological payloads upon binding to that tissue.

The experimental procedure for creating DREADDs is reported to be fairly straightforward⁵⁰. In short, the receptor of choice is randomly mutated to create a library of many different mutants, test the interactions of CNO with new mutant receptors, select mutants that are capable of binding to CNO, go through more rounds of random mutation to select for mutants that have even better interactions with CNO, select the best mutant candidate to be expressed in mammalian cells to then perform the binding assay⁵⁰.

If it is not possible to get GPCR expression in yeast, an expression plasmid with a high copy number will be selected. Starting with high copy number plasmid overexpressing the GPCR could be toxic to yeast and will result in greater variation in copy number among cells, resulting in different expression levels of GPCR from colony to colony and decreasing experimental reproducibility. Lastly, multiple yeast strains will be used to maximize the directed mutagenesis approaches.

1. A nucleic acid construct comprising: a first genetic circuit comprising a tissue-specific promoter operatively linked to a sequence capable of encoding a modified receptor.

2. The nucleic acid construct of claim 1, wherein the tissue-specific promoter is CXCL4, GPIIb, or PTPRC.

3.-5. (canceled)

6. The nucleic acid construct of claim 1, wherein the modified receptor is a modified G-protein coupled receptor (GPCR) or a modified protease-activated receptor (PAR).

7. The nucleic acid construct of claim 6, wherein the modified GPCR is a Gq, a Gi, a Gs or a G₁₂/G₁₃ GPCR.

8. The nucleic acid construct of claim 6, wherein the modified PAR is PAR1, PAR2, PAR3 or PAR4.

9. The nucleic acid construct of claim 1, further comprising a second genetic circuit, wherein the second genetic circuit comprises one or more megakaryocyte differentiation genes.

10. The nucleic acid construct of claim 9, wherein the one or more megakaryocyte differentiation genes are HoxB4, GATA1, c-MYC, BMI1, BCL-XL, PLK-1 or a combination thereof.

11.-15. (canceled)

16. The nucleic acid construct of claim 9, wherein the first genetic circuit or the second genetic circuit further comprises a gene of interest.

17. The nucleic acid construct of claim 16, wherein the gene of interest is a therapeutic agent.

18. (canceled)

19. (canceled)

20. A pluripotent stem cell or a megakaryocyte comprising the nucleic acid construct of claim 1.

21.-39. (canceled)

40. An engineered platelet comprising a modified receptor, wherein the modified receptor is a modified G-protein coupled receptor (GPCR) or a modified protease-activated receptor (PAR).

41. (canceled)

42. The engineered platelet of claim 40, wherein the modified GPCR is a Gq, a Gi, a Gs or a G₁₂/G₁₃ GPCR.

43. The engineered platelet of claim 40, wherein the modified PAR is PAR1, PAR2, PAR3 or PAR4.

44. The engineered platelet of claim 40, further comprising a therapeutic agent.

45. A method of producing a platelet or a population of platelets comprising a modified receptor, the method comprising:
a. providing pluripotent stem cells comprising any of the nucleic acid constructs of claim 1;
b. culturing the pluripotent stem cells in a media under conditions to permit the expansion of the pluripotent stem cells to megakaryocytes; and
c. differentiating the megakaryocytes into platelets;
wherein the platelets or the population of platelets comprise the modified receptor.

46.-56. (canceled)

57. A method of treating a human patient, the method comprising: a) administering one or more of the platelets of claim 40 to the human patient; and b) administering an exogenous agonist to the human patient; wherein the presence of the exogenous agonist activates the modified receptor.

58. (canceled)

59. The method of claim 57, wherein the human patient has cancer, diabetes, or lysosomal storage disease.

60.-63. (canceled)

64. The method of claim 57, wherein the activation of the modified receptor induces the release of the therapeutic agent from the platelet of claim 44 or one or more endogenous biomolecules.

65. The method of claim 57, wherein the one or more platelets or the population of platelets is administered via intravenous injection or transfusion.

66. The method of claim 57, wherein the exogenous agonist is administered via intracranial, instraspinal, intramuscular, or intravenous injection or orally.

67.-71. (canceled)


SEQUENCE LISTING (1 sequence)

SEQ ID NO: 1 - protein, 4 aa, Artificial Sequence
  Synthetic construct
LKNG